(19) United States
(12) Patent Application Publication
(10) Pub. No.: US 2025/0015550 A1
(43) Pub. Date: Jan. 9, 2025

(54) Laser Generator with Enhanced Frequency Rate

(71) Applicant: Lumenis Ltd., Yokneam Ilit (IL)

(72) Inventors: Arkady Khachaturov, Kiryat Yam (IL); Tal Waisman, Haifa (IL)

(73) Assignee: Lumenis Ltd., Yokneam llit (IL)

(21) Appl. No.: 18/764,762

(22) Filed: Jul. 5, 2024

Related U.S. Application Data
(60) Provisional application No. 63/525,251, filed on Jul. 6, 2023.

(51) Int. Cl. H01S 3/00

(52) U.S. Cl. CPC H01S 3/0057; H01S 3/0085

(57) ABSTRACT

The disclosure provides a laser generator comprising several laser sources where each laser source is arranged to generate a series of laser pulses offset in time from the other series of laser pulses generated by the other laser sources. The offset series of pulses are combined to provide a composite series of laser pulses with a higher frequency than each of the series of pulses have individually. Further, the present disclosure provides that the pulses of the composite series of laser pulses are non-equidistant from each other.

### CROSS-REFERENCE TO RELATED APPLICATIONS

This application claims the benefit of U.S. Provisional Patent Application Ser. No. 63/525,251 filed on Jul. 6, 2023, the disclosure of which is incorporated herein by reference.

### TECHNICAL FIELD

The present disclosure generally relates to medical laser systems and particularly, but not exclusively, to increasing the frequency of generated laser pulses.

### BACKGROUND

Medical lasers are used in a variety of procedures to provide laser energy, which laser energy is directed towards a target, often using a fiber as a conduit for the laser energy. One such procedure, to address renal calculi (e.g., kidney stones) is ureteral endoscopy, or lithotripsy. An endoscopic probe, with a camera or other sensor, is inserted into the patient's urinary tract to locate the calculi for removal. In endoscopic lithotripsy, the probe also includes an optical fiber, which conducts a laser beam to disintegrate the calculi as they are found.

Holmium:yttrium-aluminum-garnet (Ho:YAG) lasers have been favored for the treatment of urinary calculus since shortly after their introduction in the 1990s as the generated laser energy can fragment all calculus compositions and often produces less calculus migration (retropulsion) during treatment than the short-pulsed lasers. However, conventional Ho:YAG laser generators have a practical limit to the frequency with which they can generate laser pulses. For example, as the frequency increases, the effectiveness of the laser resonator decreases. Further, increasing the frequency requires a corresponding increase in the cooling apparatus and mechanism for the resonator.

### BRIEF SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

The disclosure provides a laser generator comprising several laser sources where each laser source is arranged to generate a series of laser pulses offset in time from the other series of laser pulses generated by the other laser sources. The offset series of pulses are combined, for example, with a rotating mirror to provide a composite series of laser pulses with a higher frequency than each of the series of pulses have individually.

Further, of note, the present disclosure provides that each laser source can be configured to generate equidistant pulses, or rather, the series of laser pulses generated by each source comprises a repeating series of two pulses where the delay between the pulses in the repeating series of two pules is less than the delay between each series of two pulses. This is described in greater detail below.

In some embodiments, the disclosure can be implemented as a method for a laser generator. The method can comprise sending, from a processor to a first laser source of the laser generator, a first control signal comprising an indication to generate a first repeating series of two laser pulses; sending, from the processor to a second laser source of the laser generator, a second control signal comprising an indication to generate a second repeating series of two laser pulses, the second repeating series of two laser pulses temporally offset from the first repeating series of two laser pulses; and combining the first repeating series of two laser pulses and the second repeating series of two laser pulses to form a composite series of laser pulses.

With further embodiments, the method can comprise sending, from the processor to a third laser source of the laser generator, a third control signal comprising an indication to generate a third repeating series of two laser pulses, the third repeating series of two laser pulses temporally offset from both the first repeating series of two laser pulses and the second repeating series of two laser pulses; and combining the first repeating series of two laser pulses, the second repeating series of two laser pulses, and the third repeating series of two laser pulses to form the composite series of laser pulses.

With further embodiments, the method can comprise sending, from the processor to a fourth laser source of the laser generator, a fourth control signal comprising an indication to generate a fourth repeating series of two laser pulses, the fourth repeating series of two laser pulses temporally offset from the first repeating series of two laser pulses, the second repeating series of two laser pulses, and the third repeating series of two laser pulses; and combining the first repeating series of two laser pulses, the second repeating series of two laser pulses, the third repeating series of two laser pulses, and the fourth repeating series of two laser pulses to form the composite series of laser pulses.

With further embodiments of the method, a delay between each series of two laser pulses in the first repeating series of two laser pulses, the second repeating series of two laser pulses, the third repeating series of two laser pulses, and the fourth repeating series of two laser pulses equals approximately 66.68 milliseconds (ms).

With further embodiments of the method, a delay between each laser pulse of each series of two laser pulses in the first repeating series of two laser pulses, the second repeating series of two laser pulses, the third repeating series of two laser pulses, and the fourth repeating series of two laser pulses is less than or equal to 8 ms.

With further embodiments of the method, the second repeating series of two laser pulses is delayed from the first repeating series of two laser pulses by approximately 16.67 ms, wherein the third repeating series of two laser pulses is delayed from the second repeating series of two laser pulses by approximately 16.67 ms, and wherein the fourth repeating series of two laser pulses is delayed from the third repeating series of two laser pulses by approximately 16.67 ms.

With further embodiments of the method, a delay between each series of two laser pulses in the first repeating series of two laser pulses and the second repeating series of two laser pulses is greater than or equal to 50 milliseconds (ms).

With further embodiments of the method, a delay between each laser pulse of each series of two laser pulses in the first repeating series of two laser pulses and the second repeating series of two laser pulses is less than or equal to 10 ms.

With further embodiments of the method, the second repeating series of two laser pulses is delayed from the first repeating series of two laser pulses by greater than or equal to 12.5 ms.

In some embodiments, the disclosure can be implemented as an apparatus, which can comprise a plurality of laser sources, each arranged to output a pulsed laser beam; a beam combiner apparatus arranged to combine the pulsed laser beams output from the plurality of laser sources into a composite pulsed laser beam; and a laser controller coupled to the plurality of laser sources and the beam combiner, the controller configured to: send to a first laser source of the plurality of laser sources, a first control signal comprising an indication to generate the pulsed laser beam comprising a first repeating series of two laser pulses; and send to a second laser source of the plurality of laser sources, a second control signal comprising an indication to generate the pulsed laser beam comprising a second repeating series of two laser pulses, the second repeating series of two laser pulses temporally offset from the first repeating series of two laser pulses such that the composite pulsed laser beam comprises the first repeating series of two laser pulses and the second repeating series of two laser pulses.

With further embodiments of the apparatus, a delay between each series of two laser pulses in the first repeating series of two laser pulses and the second repeating series of two laser pulses is greater than or equal to 50 milliseconds (ms).

With further embodiments of the apparatus, a delay between each laser pulse of each series of two laser pulses in the first repeating series of two laser pulses and the second repeating series of two laser pulses is less than or equal to 10 ms.

With further embodiments of the apparatus, the second repeating series of two laser pulses is delayed from the first repeating series of two laser pulses by greater than or equal to 12.5 ms.

With further embodiments of the apparatus, the controller can be configured to send to a third laser source of the plurality of laser sources, a third control signal comprising an indication to generate the pulsed laser beam comprising a third repeating series of two laser pulses, the third repeating series of two laser pulses temporally offset from both the first repeating series of two laser pulses and the second repeating series of two laser pulses; and send to a fourth laser source of the plurality of laser sources, a fourth control signal comprising an indication to generate the pulsed laser beam comprising a fourth repeating series of two laser pulses, the fourth repeating series of two laser pulses temporally offset from the first repeating series of two laser pulses, the second repeating series of two laser pulses, and the third repeating series of two laser pulses such that the composite pulsed laser beam comprises the first repeating series of two laser pulses, the second repeating series of two laser pulses, the third repeating series of two laser pulses, and the fourth repeating series of two laser pulses.

With further embodiments of the apparatus, a delay between each series of two laser pulses in the first repeating series of two laser pulses, the second repeating series of two laser pulses, the third repeating series of two laser pulses, and the fourth repeating series of two laser pulses equals approximately 66.68 milliseconds (ms), wherein a delay between each laser pulse of each series of two laser pulses in the first repeating series of two laser pulses, the second repeating series of two laser pulses, the third repeating series of two laser pulses, and the fourth repeating series of two laser pulses is less than or equal to 8 ms, wherein the second repeating series of two laser pulses is delayed from the first repeating series of two laser pulses by approximately 16.67 ms, wherein the third repeating series of two laser pulses is delayed from the second repeating series of two laser pulses by approximately 16.67 ms, and wherein the fourth repeating series of two laser pulses is delayed from the third repeating series of two laser pulses by approximately 16.67 ms.

In some embodiments, the disclosure can be implemented as an apparatus for a medical laser console, which can comprise a plurality of laser sources, each arranged to output a pulsed laser beam; a beam combiner apparatus arranged to combine the pulsed laser beams output from the plurality of laser sources into a composite pulsed laser beam; and a laser controller coupled to the plurality of laser sources and the beam combiner, the controller configured to send to a first laser source of the plurality of laser sources, a first control signal comprising an indication to generate the pulsed laser beam comprising a first repeating series of two laser pulses; and send to a second laser source of the plurality of laser sources, a second control signal comprising an indication to generate the pulsed laser beam comprising a second repeating series of two laser pulses, the second repeating series of two laser pulses temporally offset from the first repeating series of two laser pulses such that the composite pulsed laser beam comprises the first repeating series of two laser pulses and the second repeating series of two laser pulses.

With further embodiments of the apparatus, a delay between each series of two laser pulses in the first repeating series of two laser pulses and the second repeating series of two laser pulses is greater than or equal to 50 milliseconds (ms).

With further embodiments of the apparatus, a delay between each laser pulse of each series of two laser pulses in the first repeating series of two laser pulses and the second repeating series of two laser pulses is less than or equal to 10 ms.

With further embodiments of the apparatus, the second repeating series of two laser pulses is delayed from the first repeating series of two laser pulses by greater than or equal to 12.5 ms.

With further embodiments of the apparatus, the controller can be configured to send to a third laser source of the plurality of laser sources, a third control signal comprising an indication to generate the pulsed laser beam comprising a third repeating series of two laser pulses, the third repeating series of two laser pulses temporally offset from both the first repeating series of two laser pulses and the second repeating series of two laser pulses, such that the composite pulsed laser beam comprises the first repeating series of two laser pulses, the second repeating series of two laser pulses, the third repeating series of two laser pulses.

With further embodiments of the apparatus, the controller can be configured to send to a fourth laser source of the plurality of laser sources, a fourth control signal comprising an indication to generate the pulsed laser beam comprising a fourth repeating series of two laser pulses, the fourth repeating series of two laser pulses temporally offset from the first repeating series of two laser pulses, the second repeating series of two laser pulses, and the third repeating series of two laser pulses such that the composite pulsed laser beam comprises the first repeating series of two laser pulses, the second repeating series of two laser pulses, the third repeating series of two laser pulses, and the fourth repeating series of two laser pulses.

With further embodiments of the apparatus, a delay between each series of two laser pulses in the first repeating series of two laser pulses, the second repeating series of two laser pulses, the third repeating series of two laser pulses, and the fourth repeating series of two laser pulses is between 57.1 milliseconds (ms) and 88.9 ms.

With further embodiments of the apparatus, a delay between each laser pulse of each series of two laser pulses in the first repeating series of two laser pulses, the second repeating series of two laser pulses, the third repeating series of two laser pulses, and the fourth repeating series of two laser pulses is less than or equal to 9.7 ms or is between 1.8 ms and 9.7 ms.

With further embodiments of the apparatus, the second repeating series of two laser pulses is delayed from the first repeating series of two laser pulses by approximately 16.67 ms, wherein the third repeating series of two laser pulses is delayed from the second repeating series of two laser pulses by approximately 16.67 ms, and wherein the fourth repeating series of two laser pulses is delayed from the third repeating series of two laser pulses by approximately 16.67 ms.

In some embodiments, the disclosure can be implemented as a computer readable storage device for a medical laser console controller, comprising instructions which when executed by circuitry of the medical laser console controller cause the medical laser console to send to a first laser source of the medical laser console, a first control signal comprising an indication to generate a first repeating series of two laser pulses; and send to a second laser source of the medical laser console, a second control signal comprising an indication to generate a second repeating series of two laser pulses, the second repeating series of two laser pulses temporally offset from the first repeating series of two laser pulses, wherein the first repeating series of two laser pulses and the second repeating series of two laser pulses are combined to form a composite series of laser pulses.

With further embodiments of the storage device, the instructions when executed by the circuitry further cause the medical laser console to send to a third laser source of the laser generator, a third control signal comprising an indication to generate a third repeating series of two laser pulses, the third repeating series of two laser pulses temporally offset from both the first repeating series of two laser pulses and the second repeating series of two laser pulses, wherein the first repeating series of two laser pulses, the second repeating series of two laser pulses, and the third repeating series of two laser pulses are combined to form the composite series of laser pulses.

With further embodiments of the storage device, the instructions when executed by the circuitry further cause the medical laser console to send to a fourth laser source of the laser generator, a fourth control signal comprising an indication to generate a fourth repeating series of two laser pulses, the fourth repeating series of two laser pulses temporally offset from the first repeating series of two laser pulses, the second repeating series of two laser pulses, and the third repeating series of two laser pulses, wherein the first repeating series of two laser pulses, the second repeating series of two laser pulses, the third repeating series of two laser pulses, and the fourth repeating series of two laser pulses are combined to form the composite series of laser pulses.

With further embodiments of the storage device, a delay between each series of two laser pulses in the first repeating series of two laser pulses, the second repeating series of two laser pulses, the third repeating series of two laser pulses, and the fourth repeating series of two laser pulses is less than or equal to 100 milliseconds (ms).

With further embodiments of the storage device, a delay between each laser pulse of each series of two laser pulses in the first repeating series of two laser pulses, the second repeating series of two laser pulses, the third repeating series of two laser pulses, and the fourth repeating series of two laser pulses is less than or equal to 9.7 ms.

With further embodiments of the storage device, the second repeating series of two laser pulses is delayed from the first repeating series of two laser pulses by approximately 16.67 ms, wherein the third repeating series of two laser pulses is delayed from the second repeating series of two laser pulses by approximately 16.67 ms, and wherein the fourth repeating series of two laser pulses is delayed from the third repeating series of two laser pulses by approximately 16.67 ms.

With further embodiments of the storage device, a delay between each series of two laser pulses in the first repeating series of two laser pulses and the second repeating series of two laser pulses is greater than or equal to 50 milliseconds (ms), wherein a delay between each laser pulse of each series of two laser pulses in the first repeating series of two laser pulses and the second repeating series of two laser pulses is less than or equal to 10 ms, and wherein the second repeating series of two laser pulses is delayed from the first repeating series of two laser pulses by greater than or equal to 12.5 ms.

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1A illustrates an example medical laser system in accordance with at least one embodiment of the disclosure.

FIG. 1B and FIG. 1C illustrate a portion of the example medical laser system of FIG. 1A in more detail.

FIG. 2 illustrates an example timing diagram of pulses of laser energy in accordance with at least one embodiment of the disclosure.

FIG. 3 illustrates a method for a medical laser system in accordance with at least one embodiment of the disclosure.

FIG. 4 illustrates another example timing diagram of pulses of laser energy in accordance with at least one embodiment of the disclosure.

FIG. 5 illustrates a graph showing laser pulse versus laser pump energy in accordance with at least one embodiment of the disclosure.

FIG. 6 illustrates another example timing diagram in accordance with at least one embodiment of the disclosure.

### DETAILED DESCRIPTION

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

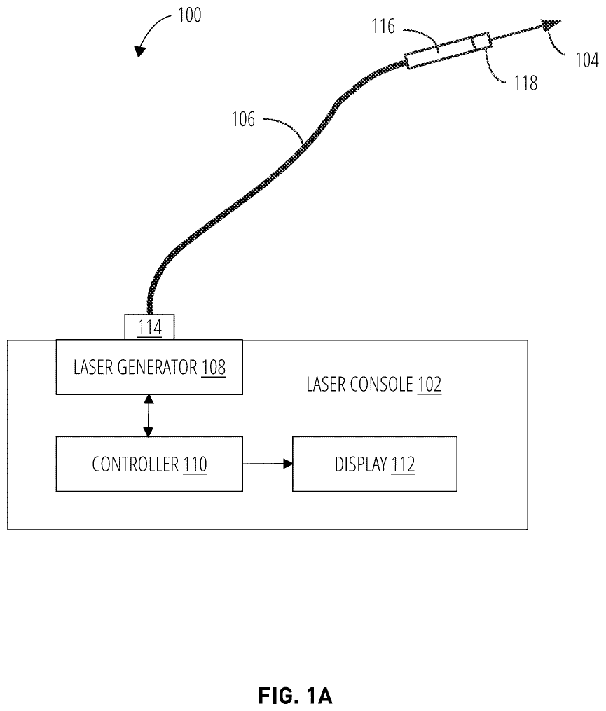

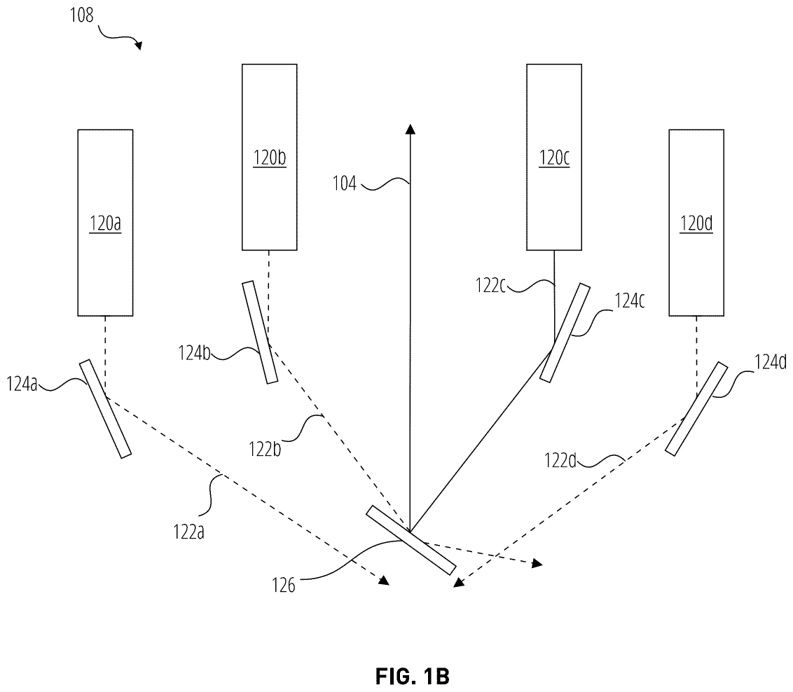

FIG. 1A illustrates a medical laser system 100, according to embodiments of the present disclosure. In some embodiments, the medical laser system 100 can be configured to perform laser lithotripsy procedures. The medical laser system 100 can include a laser console 102 arranged to generate a laser energy 104 and deliver to laser energy to a target (not shown) via an optical fiber 106. The laser console 102 can include a laser generator 108, controller 110, and display 112. In general, the laser generator 108 comprises several laser sources (e.g., see FIG. 1B and FIG. 1C) arranged to generate laser pulse trains and optical components to combine the laser pulse trains into laser energy 104 that includes pulses from each of the pulse trains generated by the several laser sources. This is described in greater detail below.

The controller 110 can comprise circuitry (e.g., programmable logic, or the like) or processing components and memory storing instructions executable by the processing components configured to control the laser generator 108 and/or generate graphical elements comprising indications of operation or performance of laser generator 108 and cause the graphical elements to be displayed on display 112.

The optical fiber 106 can be coupled to laser generator 108 via a coupler 114. Further, the optical fiber 106 can comprise a probe 116 having a distal end 118. The probe 116 can be inserted into a working channel of an endoscope (e.g., a ureteroscope, or the like) and the distal end 118 extended to protrude from the distal end of the endoscope such that laser energy 104 can be directed at a target (e.g., stone, tissue, or the like).

Controller 110 can further be configured and/or provided with input devices (not shown) such that controller can receive indications of operation conditions or parameters (e.g., from a user, or the like). Responsive to receiving operating parameters, controller 110 can cause laser generator 108 to generate laser energy 104 having characteristics based on the received operating parameters (e.g., magnitude of pulses, pulse width, wavelength, duration of pulses, frequency of pulses, or the like). More particularly, controller 110 can cause individual laser sources in laser generator 108 to generate pulses of laser energy and can combine the pulses of laser energy from each laser source to from laser energy 104.

FIG. 1B and FIG. 1C illustrate an example of laser generator 108 having four (4) laser sources, or laser resonators 120a, 120b, 120c, and 120d. Each of laser resonators 120a, 120b, 120c, and 120d comprise a lasing medium (e.g., Ho:YAG, Thulium, etc.), optical pumping components, cooling components. It is to be appreciated that as the frequency of pulses generated by a lasing medium in one of laser resonators 120a, 120b, 120c, and 120d increases, the effectiveness of the lasing medium decreases. As such, controller 110 can control laser resonators 120a, 120b, 120c, and 120d of laser generator 108 and cause the laser resonators 120a, 120b, 120c, and 120d to generate series of laser pulses 122a, 122b, 122c, and 122d, respectively. Each series of laser pulses 122a, 122b, 122c, and 122d is generated offset in time from each other. Further, each series of series of laser pulses 122a, 122b, 122c, and 122d comprises a series of two pulses followed by a longer delay in which the other series of laser pulses are generated.

The laser generator 108 further includes optical components arranged to combine the series of laser pulses 122a, 122b, 122c, and 122d into laser energy 104. For example, laser generator 108 can comprise mirrors 124a, 124b, 124c, and 124d arranged to direct series of laser pulses 122a, 122b, 122c, and 122d to rotating mirror 126. The rotating mirror 126 is arranged to move to combine the series of laser pulses 122a, 122b, 122c, and 122d to form laser energy 104 and direct laser energy 104 towards coupler 114. For example, in a first position depicted in FIG. 1B, rotating mirror 126 can be configured to direct series of laser pulses 122c generated by laser resonator 120c along an output path and form laser energy 104.

FIG. 1C depicts rotating mirror 126 in another, second position, in which rotating mirror 126 is configured to direct series of laser pulses 122b generated by laser resonator 120b along the output path and form laser energy 104. In such a manner, laser energy 104 can be formed to include laser pulses generated by all the laser sources. It is to be appreciated that laser generator 108 can further includes other optical components arranged to collimate and/or couple laser energy 104 with coupler 114.

Further, it is to be appreciated that a variety of optical apparatus and configurations can be provided where several laser sources (e.g., laser resonators 120a, 120b, 120c, and 120d) can be configured to generate a series of laser pulses each temporally separated from the other and optically combined to form a composite series of laser pulses (e.g., laser energy 104). For example, International Application No. PCT/IL2019/051060, filed on Sep. 25, 2019, which application is incorporated herein by reference in its entirety, describes a laser apparatus including multiple laser devices arranged to emit laser pulses and a motorized laser pulse reflection apparatus arranged to be continuously rotated at a uniform angular velocity for reflecting laser pulses along a single optical path towards a target. The application further describes a controller synchronized with the motorized laser pulse reflection apparatus for individually firing the multiple laser devices for emitting a train of laser pulses reaching the target without obstruction by the motorized laser pulse reflection arrangement. The devices and arrangements described in the above referenced International application can be implemented by embodiments of the present disclosure without departing from the scope of the claims. In such a manner, the frequency of the composite series of laser pulses can be increased above what each individual laser source can generate on its own.

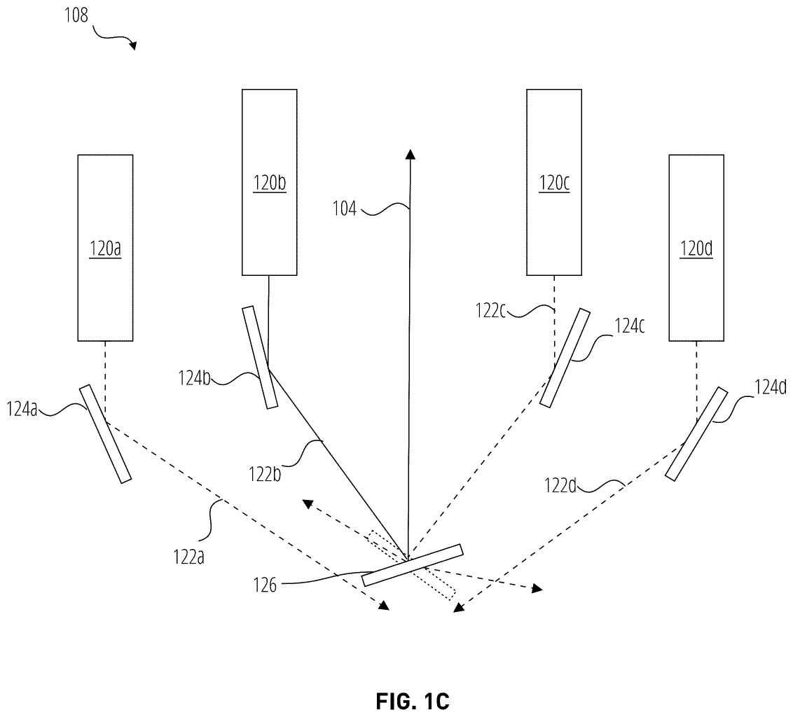

FIG. 2 illustrates a timing diagram 200 showing the series of laser pulses 122a, 122b, 122c, and 122d. Further, timing diagram 200 depicts the laser energy 104 that is formed from the series of laser pulses 122a, 122b, 122c, and 122d. As depicted, each of the series of laser pulses 122a, 122b, 122c, and 122d comprises a repeating series of two laser pulses 202a, 202b, 202c, and 202d, respectively, that are offset in time from each other. Each pulse of the series repeating series of two laser pulses 202a, 202b, 202c, and 202d is separated by a delay 204. For example, the first pulse 212a is separated from the second pulse 212b by delay 204. Similarly, each series of two laser pulses 202a, 202b, 202c, and 202d is separated by a delay 206. For example, series of laser pulses 202a is separated from the repeating series of laser pulses 202a by delay 206.

Accordingly, each laser source (e.g., laser resonators 120a, 120b, 120c, and 120d) is configured to generate 2 pulses (e.g., laser pulses 202a, 202b, 202c, and 202d) when rotating mirror 126 is in a corresponding position. For example, laser resonator 120b is configured to generate laser pulses 202b when rotating mirror 126 is in the position depicted in FIG. 1C.

In some embodiments, rotating mirror 126 is configured to rotate at a constant velocity. With other embodiments, rotating mirror 126 is configured to move (e.g., with maximal possible acceleration, or the like) and stop at several (e.g., four, the number of laser resonators, or the like) predefined locations associated with each of the laser resonator. When rotating mirror 126 is at each of the pre-defined locations, controller 110 can be configured to cause the laser sources of laser generator 108 (e.g., laser resonators 120a, 120b, 120c, and 120d) to generate individual series of laser pulses (e.g., series of laser pulses 122a, 122b, 122c, and 122d) in time with the rotation or movement of rotating mirror 126 such that laser energy 104 is formed and directed to a single point (e.g., coupler 114, or the like). In such a manner, laser energy 104 is formed to include laser pulses 202a, 202b, 202c, and 202d where delay 208 separates each individual series of laser pulses 202a, 202b, 202c, and 202d. With some embodiments, laser resonators 120a, 120b, 120c, and 120d are configured to generate laser pulses 202a, 202b, 202c, and 202d where delay 204 is the same as delay 208. It will be appreciated that although this provides for an increase in the frequency of the laser energy 104 without increasing the acceleration of rotating mirror 126, the cooling requirement for each of laser resonators 120a, 120b, 120c, and 120d will increase (e.g., double).

In some examples, delay 206, or rather each of laser pulses 202a, 202b, 202c, and/or 202d, respectively, can be separated by between 50 and 66.68 milliseconds (ms), by greater than or equal to 50 ms, by greater than or equal to 66.68 ms, or by between 57.1 ms and 88.9 ms. Additionally, each laser pulse in a respective series of two laser pulses (e.g., first pulse 212a and second pulse 212b of laser pulses 202a or the like) can be separated by delay 204, where delay 204 can be between 3 ms and 10 ms, by approximately 8.33 ms, by approximately 6.25 ms, by between 6.25 and 8.33 ms, by between 3 and 8 ms, or by between 1.8 ms and 9.7 ms.

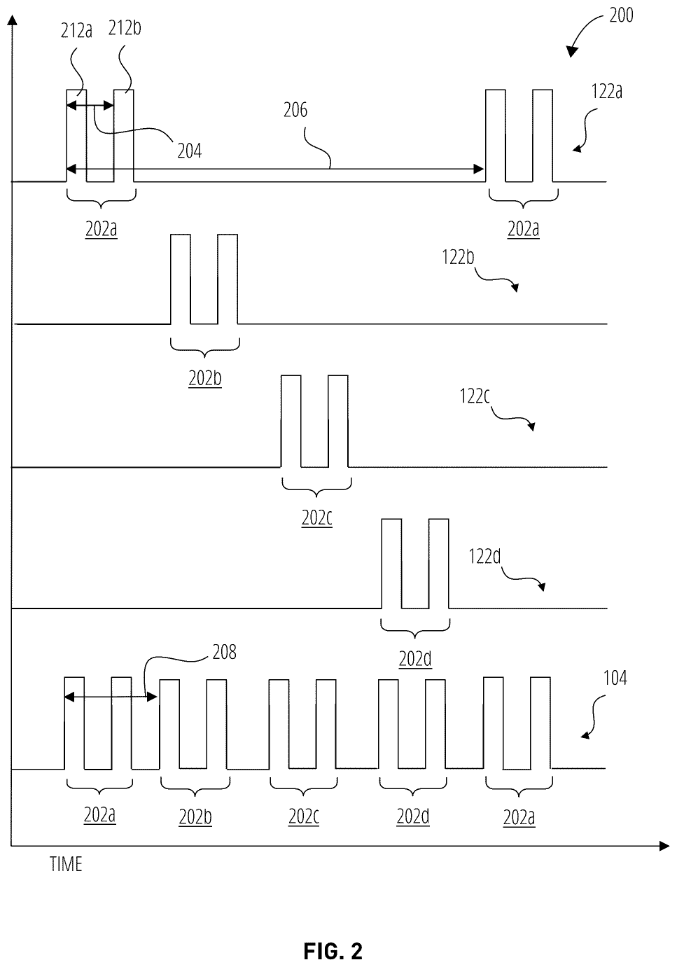

FIG. 3 illustrates a method 300 for a laser generator, according to embodiments of the present disclosure. The method 300 can be implemented by medical laser system 100 and particularly laser generator 108 to provide laser energy 104 having a higher frequency than conventional laser generators can provide. Method 300 can begin at block 302. At block 302 “send, from a controller to a first laser source of a laser generator, a first control signal comprising an indication to generate a first repeating series of two laser pulses” a first control signal is sent from a controller to a first laser source where the first control signal comprises an indication to generate a first repeating series of two laser pulses. For example, controller 110 can send a control signal to laser resonator 120a to cause laser resonator 120a to generate series of laser pulses 122a having repeating series of laser pulses 202a.

Continuing to block 304 “send, from the controller to a second laser source of the laser generator, a second control signal comprising an indication to generate a second repeating series of two laser pulses, the second repeating series of two laser pulses temporally offset from the first repeating series of two laser pulses” a second control signal is sent from the controller to a second laser source where the second control signal comprises an indication to generate a second repeating series of two laser pulses that are temporally offset (or delayed) from the first repeating series of two laser pulses. For example, controller 110 can send a control signal to laser resonator 120b to cause laser resonator 120b to generate series of laser pulses 122b having repeating series of laser pulses 202b, which are delayed or temporally offset from laser pulses 202a by a delay 208.

Continuing to block 306 “combine the first repeating series of two laser pulses and the second repeating series of two laser pulses to form a composite series of laser pulses” the first and second repeating series of two laser pulses can be combined to form a composite series of laser pulses. For examples, series of laser pulses 122a and series of laser pulses 122b can be combined to form laser energy 104 having both laser pulses 202a and 202b.

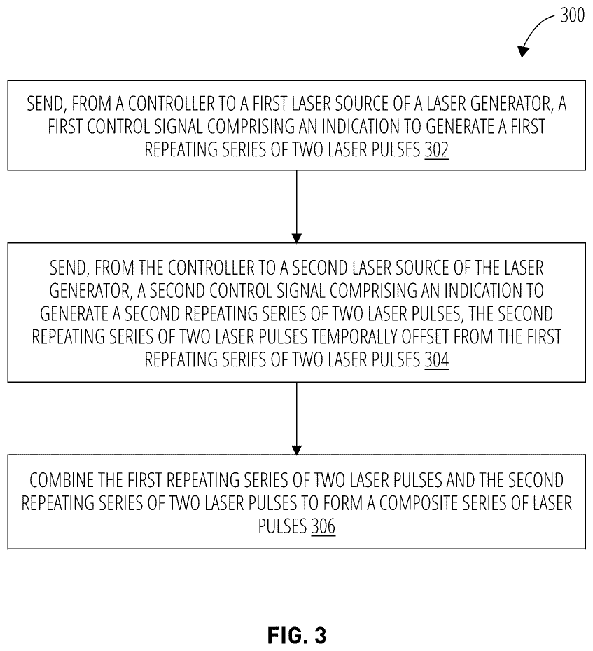

With some embodiments, the delay 208 is not a multiple of the delay 204. Or rather, with some embodiments, the pulses in the composite series of laser pulses (e.g., laser energy 104) are not equidistant from each other. For example, FIG. 4 illustrates a timing diagram 400 showing a composite laser beam 402 having non-equidistant pulses. Composite laser beam 402 can be formed from series of laser pulses 404a and 404b where series of laser pulses 404a and 404b each include a repeating series of two laser pulses 406a and 406b, respectively. The laser pulses 408 in each repeating series of two laser pulses 406a and 406b can be separated by a delay 410 while each repeating series of two laser pulses 406a and 406b can themselves be separated by a delay 412. Likewise, repeating series of two laser pulses 406a can be separated from repeating series of two laser pulses 406b in composite laser beam 402 by delay 414.

As depicted in FIG. 4, delay 414 is not a multiple of delay 410, or rather, the laser pulses 408 in composite laser beam 402 are not equidistant from each other. For example, in some embodiments, composite laser beam 402 can be configured to have a frequency of 120 Hertz (Hz). As such, delay 414 can be 16.67 ms while delay 410 can be less than or equal to 10 ms. With some embodiments, delay 410 can be approximately 3 ms. Controller 110 can be configured to send control signals to laser generator 108 and particularly to laser sources, or laser resonators in laser generator 108 to cause laser generator 108 to generate laser energy 104 having non-equidistant laser pulses as depicted in FIG. 4.

Such approach provides that individual laser sources (e.g., laser resonators 120a, 120b, 120c, and 120d) within laser generator 108 need not have significantly increased cooling capacity to increase the frequency of laser energy 104. Where the delay between two pulses from the series of two pulses (e.g., delay 204, delay 410, or the like) is less than luminescence decay time for the lasing medium residual pump energy from the first pulse remains within the lasing medium. Therefore, less pump energy is required for the second pulse than was required for the first pulse. For example, a Cr.Tm.Ho:YAG (DTH:YAG) crystal has a decay time of approximately 10 ms. As such, where delay 204 or delay 410 is less than 10 ms, the pump energy required for the second pulse in the series of two pulses will be less than was required for the first pulse.

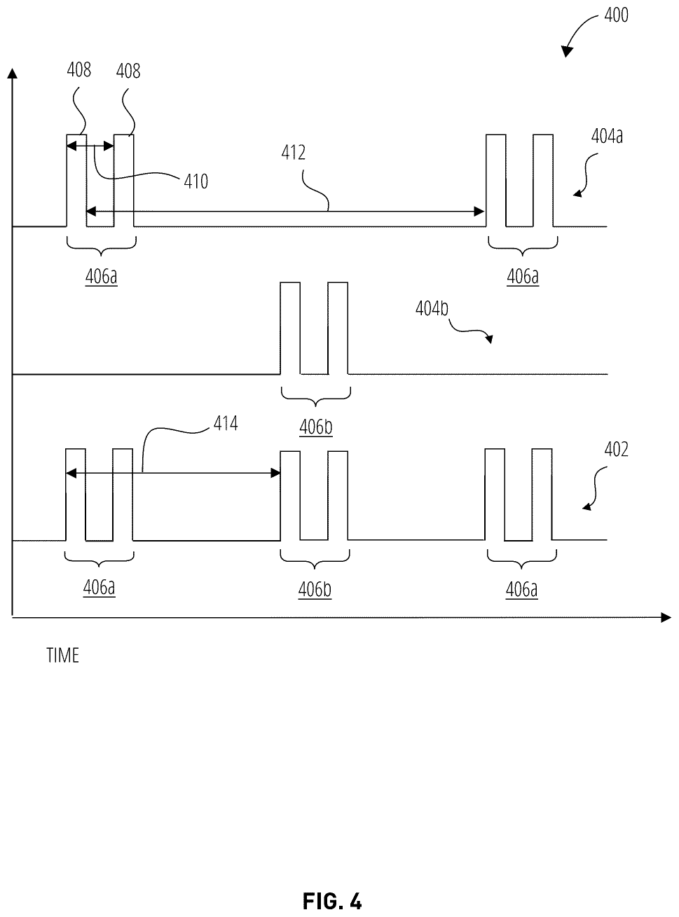

FIG. 5 illustrates graph 500 showing experimental results for generating a 120 Hz frequency composite laser beam from four (4) laser sources. In the example, each source operated at 15 Hz and generated 2 pulses per time period for an effective frequency of 30 Hz per laser source. The rotating mirror (e.g., rotating mirror 126) operated at 60 Hz, for an overall frequency of 120 Hz for the composite laser beam. As can clearly be seen from graph 500, each lasing makes possible to save ΔE energy. As shown by Table 1 shown below, which references obtained experimental results, the saved energy value depends on the delay time t (e.g., delay 204, delay 410, etc.) where the longer the delay time t, the smaller the energy gain.

TABLE 1
Repetition Rate for 2 pulses, 15 Hz (60 Hz/4)

| t in microseconds (μs) | Δ in Jules (J) |
|---|---|
| 3200 | 6 |
| 3000 | 6.5 |
| 2500 | 7.5 |
| 2000 | 8.5 |
| 1500 | 10 |

Table 2 and Table 3 shown below reference saved energy values for experimental results obtained using alternative repetition rates to those shown in graph 500 and Table 1. However, as can be seen, these other experiments confirm that the saved energy values depend on the delay time between pulses in each repeating series of two pulses.

TABLE 2
Repetition Rate for 2 pulses, 14 Hz (56 Hz/4)

| t in microseconds (μs) | Δ in Jules (J) |
|---|---|
| 4500 | 4 |
| 4300 | 5 |
| 3000 | 7 |
| 2000 | 9 |
| 1500 | 10 |

TABLE 3
Repetition Rate for 2 pulses, 12 Hz (48 Hz/4)

| t in microseconds (μs) | Δ in Jules (J) |
|---|---|
| 7000 | 3 |
| 6000 | 4 |
| 5000 | 5 |
| 4000 | 6 |
| 3000 | 7 |

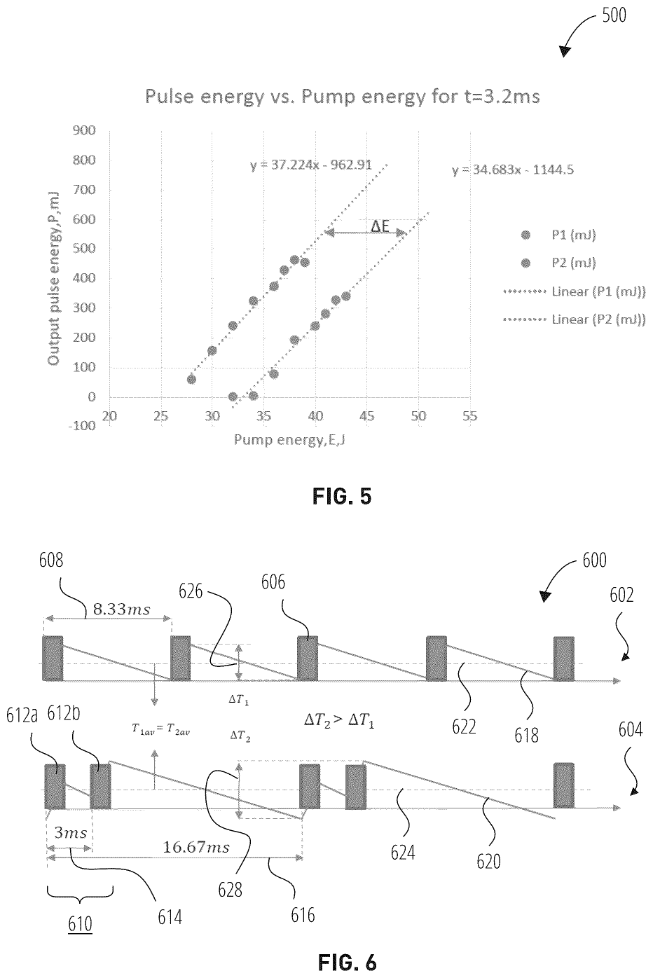

FIG. 6 illustrates a timing diagram 600 showing an equidistant pulsed laser beam 602 and a non-equidistant pulsed laser beam 604 both having the same frequency, which is 120 Hz in this example. The equidistant pulsed laser beam 602 comprises pulses 606 all separated by delay 608, thereby forming a laser beam 602 having a series of equidistantly spaced pulse 606.

Timing diagram 600 also shows non-equidistant pulsed laser beam 604 comprising a repeating series of two pulses 610 having pulse 612a and 612b. They delay between pulses 612a and 612b is 614 while the delay between the series of pulses 610 is 616. As noted above, equidistant pulsed laser beam 602 and non-equidistant pulsed laser beam 604 have a frequency of 120 Hz. However, it will be appreciated that equidistant pulsed laser beam 602 and non-equidistant pulsed laser beam 604 could have any of a variety of frequencies (e.g., between 80 and 160 Hz, or the like) and the delays would scale accordingly. Accordingly, using the example of 120 Hz, delay 608 is 8.33 ms, delay 614 is 3 ms, and delay 616 is 16.67 ms.

Timing diagram 600 further depicts temperature 618 and 620, corresponding to the temperature of equidistant pulsed laser beam 602 and non-equidistant pulsed laser beam 604, respectively. As depicted, the average of temperatures temperature 618 and temperature 620 (i.e., average temperatures 622 and 624, respectively) is the same as the equidistant pulsed laser beam 602 and non-equidistant pulsed laser beam 604 have the same average power. However, the behavior of the temperature (e.g., high and low temperature, difference between high and low temperature, etc.) differs for each beam due to the varying delays between pulses. For example, the difference between high and low temperature 626 for equidistant pulsed laser beam 602 is less than the difference between high and low temperature 628 for non-equidistant pulsed laser beam 604. This may provide a clinical advantage in that the first pulse of non-equidistant pulsed laser beam 604 (e.g., pulse 612a) may create conditions for more effective operation of the second pulse of non-equidistant pulsed laser beam 604 (e.g., pulse 612b). This can be referred to as preconditioning.

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application. When the claims use the word “or” in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

What is claimed is:

1. An apparatus comprising:
a plurality of laser sources, each arranged to output a pulsed laser beam;
a beam combiner apparatus arranged to combine the pulsed laser beams output from the plurality of laser sources into a composite pulsed laser beam; and
a laser controller coupled to the plurality of laser sources and the beam combiner, the controller configured to:
send to a first laser source of the plurality of laser sources, a first control signal comprising an indication to generate the pulsed laser beam comprising a first repeating series of two laser pulses; and
send to a second laser source of the plurality of laser sources, a second control signal comprising an indication to generate the pulsed laser beam comprising a second repeating series of two laser pulses, the second repeating series of two laser pulses temporally offset from the first repeating series of two laser pulses such that the composite pulsed laser beam comprises the first repeating series of two laser pulses and the second repeating series of two laser pulses.

2. The apparatus of claim 1, wherein a delay between each series of two laser pulses in the first repeating series of two laser pulses and the second repeating series of two laser pulses is greater than or equal to 50 milliseconds (ms).

3. The apparatus of claim 17, wherein a delay between each laser pulse of each series of two laser pulses in the first repeating series of two laser pulses and the second repeating series of two laser pulses is less than or equal to 10 ms.

4. The apparatus of claim 3, wherein the second repeating series of two laser pulses is delayed from the first repeating series of two laser pulses by greater than or equal to 12.5 ms.

5. The apparatus of claim 1, wherein the controller is configured to:
send to a third laser source of the plurality of laser sources, a third control signal comprising an indication to generate the pulsed laser beam comprising a third repeating series of two laser pulses, the third repeating series of two laser pulses temporally offset from both the first repeating series of two laser pulses and the second repeating series of two laser pulses, such that the composite pulsed laser beam comprises the first repeating series of two laser pulses, the second repeating series of two laser pulses, the third repeating series of two laser pulses.

6. The apparatus of claim 5, wherein the controller is configured to:
send to a fourth laser source of the plurality of laser sources, a fourth control signal comprising an indication to generate the pulsed laser beam comprising a fourth repeating series of two laser pulses, the fourth repeating series of two laser pulses temporally offset from the first repeating series of two laser pulses, the second repeating series of two laser pulses, and the third repeating series of two laser pulses such that the composite pulsed laser beam comprises the first repeating series of two laser pulses, the second repeating series of two laser pulses, the third repeating series of two laser pulses, and the fourth repeating series of two laser pulses.

7. The apparatus of claim 6, wherein a delay between each series of two laser pulses in the first repeating series of two laser pulses, the second repeating series of two laser pulses, the third repeating series of two laser pulses, and the fourth repeating series of two laser pulses is between 57.1 milliseconds (ms) and 88.9 ms.

8. The apparatus of claim 7, wherein a delay between each laser pulse of each series of two laser pulses in the first repeating series of two laser pulses, the second repeating series of two laser pulses, the third repeating series of two laser pulses, and the fourth repeating series of two laser pulses is less than or equal to 9.7 ms or is between 1.8 ms and 9.7 ms.

9. The apparatus of claim 6, wherein the second repeating series of two laser pulses is delayed from the first repeating series of two laser pulses by approximately 16.67 ms, wherein the third repeating series of two laser pulses is delayed from the second repeating series of two laser pulses by approximately 16.67 ms, and wherein the fourth repeating series of two laser pulses is delayed from the third repeating series of two laser pulses by approximately 16.67 ms.

10. A computer readable storage device for a medical laser console controller, comprising instructions which when executed by circuitry of the medical laser console controller cause the medical laser console to:
send to a first laser source of the medical laser console, a first control signal comprising an indication to generate a first repeating series of two laser pulses; and
send to a second laser source of the medical laser console, a second control signal comprising an indication to generate a second repeating series of two laser pulses, the second repeating series of two laser pulses temporally offset from the first repeating series of two laser pulses,
wherein the first repeating series of two laser pulses and the second repeating series of two laser pulses are combined to form a composite series of laser pulses.

11. The computer readable storage device of claim 10, wherein the instructions when executed by the circuitry further cause the medical laser console to:
send to a third laser source of the laser generator, a third control signal comprising an indication to generate a third repeating series of two laser pulses, the third repeating series of two laser pulses temporally offset from both the first repeating series of two laser pulses and the second repeating series of two laser pulses,
wherein the first repeating series of two laser pulses, the second repeating series of two laser pulses, and the third repeating series of two laser pulses are combined to form the composite series of laser pulses.

12. The computer readable storage device of claim 11, wherein the instructions when executed by the circuitry further cause the medical laser console to:
send to a fourth laser source of the laser generator, a fourth control signal comprising an indication to generate a fourth repeating series of two laser pulses, the fourth repeating series of two laser pulses temporally offset from the first repeating series of two laser pulses, the second repeating series of two laser pulses, and the third repeating series of two laser pulses,
wherein the first repeating series of two laser pulses, the second repeating series of two laser pulses, the third repeating series of two laser pulses, and the fourth repeating series of two laser pulses are combined to form the composite series of laser pulses.

13. The computer readable storage device of claim 12, wherein a delay between each series of two laser pulses in the first repeating series of two laser pulses, the second repeating series of two laser pulses, the third repeating series of two laser pulses, and the fourth repeating series of two laser pulses is less than or equal to 100 milliseconds (ms).

14. The computer readable storage device of claim 13, wherein a delay between each laser pulse of each series of two laser pulses in the first repeating series of two laser pulses, the second repeating series of two laser pulses, the third repeating series of two laser pulses, and the fourth repeating series of two laser pulses is less than or equal to 9.7 ms.

15. The computer readable storage device of claim 14, wherein the second repeating series of two laser pulses is delayed from the first repeating series of two laser pulses by approximately 16.67 ms, wherein the third repeating series of two laser pulses is delayed from the second repeating series of two laser pulses by approximately 16.67 ms, and wherein the fourth repeating series of two laser pulses is delayed from the third repeating series of two laser pulses by approximately 16.67 ms.

16. The computer readable storage device of claim 10, wherein a delay between each series of two laser pulses in the first repeating series of two laser pulses and the second repeating series of two laser pulses is greater than or equal to 50 milliseconds (ms), wherein a delay between each laser pulse of each series of two laser pulses in the first repeating series of two laser pulses and the second repeating series of two laser pulses is less than or equal to 10 ms, and wherein the second repeating series of two laser pulses is delayed from the first repeating series of two laser pulses by greater than or equal to 12.5 ms.

17. A method for a laser generator, comprising:
sending, from a processor to a first laser source of the laser generator, a first control signal comprising an indication to generate a first repeating series of two laser pulses;
sending, from the processor to a second laser source of the laser generator, a second control signal comprising an indication to generate a second repeating series of two laser pulses, the second repeating series of two laser pulses temporally offset from the first repeating series of two laser pulses; and
combining the first repeating series of two laser pulses and the second repeating series of two laser pulses to form a composite series of laser pulses.

18. The method of claim 17, further comprising:
sending, from the processor to a third laser source of the laser generator, a third control signal comprising an indication to generate a third repeating series of two laser pulses, the third repeating series of two laser pulses temporally offset from both the first repeating series of two laser pulses and the second repeating series of two laser pulses; and
combining the first repeating series of two laser pulses, the second repeating series of two laser pulses, and the third repeating series of two laser pulses to form the composite series of laser pulses.

19. The method of claim 18, further comprising:
sending, from the processor to a fourth laser source of the laser generator, a fourth control signal comprising an indication to generate a fourth repeating series of two laser pulses, the fourth repeating series of two laser pulses temporally offset from the first repeating series of two laser pulses, the second repeating series of two laser pulses, and the third repeating series of two laser pulses; and
combining the first repeating series of two laser pulses, the second repeating series of two laser pulses, the third repeating series of two laser pulses, and the fourth repeating series of two laser pulses to form the composite series of laser pulses.

20. The method of claim 19, wherein a delay between each series of two laser pulses in the first repeating series of two laser pulses, the second repeating series of two laser pulses, the third repeating series of two laser pulses, and the fourth repeating series of two laser pulses is less than or equal to 100 milliseconds (ms).
